(19) United States
(12) Patent Application Publication
(10) Pub. No.: US 2010/0241136 A1
(43) Pub. Date: Sep. 23, 2010

(54) INSTRUMENT POSITIONING/HOLDING DEVICES

(76) Inventors: Mark DOYLE, Del Mar, CA (US); Jimmy C. Caputo, Carlsbad, CA (US)

Correspondence Address: WAGNER BLECHER LLP, 123 WESTRIDGE DRIVE, WATSONVILLE, CA, 95076, US

(21) Appl. No.: 12/792,596

(22) Filed: Jun. 2, 2010

Related U.S. Application Data
(63) Continuation-in-part of application No. 12/521,073.
(60) Provisional application No. 60/872,924, filed on Dec. 5, 2006.

(51) Int. Cl. A61B 19/00

(52) U.S. Cl. 606/130

(57) ABSTRACT

Systems are provided that control the positioning of various instruments (e.g., endoscopes or tissue retractors) used during surgical procedures. A positioning mechanism holding the instrument is coupled to a control mechanism such that mechanical manipulation of the control mechanism results in movement of the positioning mechanism relative to a patient's body, thereby eliminating the need to manually hold and position the instruments.

### RELATED APPLICATIONS

This application is a continuation-in-part of pending U.S. application Ser. No. 12/521,073 entitled “Instrument Positioning/Holding Devices” which claims the benefit of U.S. Provisional Application No. 60/872,924, filed Dec. 5, 2006, which are incorporated herein by reference in its entirety.

### FIELD OF THE INVENTION

The invention relates generally to surgical instruments. More particularly, the invention relates to devices for positioning/holding a surgical instrument and methods of positioning/holding a surgical instrument.

### BACKGROUND OF THE INVENTION

Endoscopic surgical procedures are performed using long slender surgical instruments inserted into the patient through small incisions. In order to visualize the surgical site an endoscope is also inserted into the patient through another incision. A camera is attached to the endoscope, and the image is projected onto a nearby video display, which the surgeon looks at to monitor his/her activities inside the patient.

In order to permit the surgeon to use both hands for the surgery the endoscope is held in the desired position by an assistant, a stationary adjustable arm, or a voice-controlled robotic positioning device. All three have significant drawbacks. The assistant, besides being a costly paid employee, can be difficult to communicate with, can get tired, and can lose concentration and let the endoscope position drift. The stationary adjustable arms require that the surgeon reach over to adjust them with two hands, wasting valuable time and disrupting the procedure. The voice-controlled robotic positioning devices are expensive, require significant set-up effort, and often require too much time to communicate with.

During many procedures an assistant also positions and holds a retracting instrument in order to push tissue or organs out of the way of the surgeon's instrument. The same issues of communication, concentration, and fatigue are present in this task also.

There thus remains a need in the art for a positioner/holder having at least of one of the following characteristics: simple to set-up and use, controlled directly by the user, and that securely holds an endoscope and/or other instrument (hereinafter collectively referred-to as “instrument”).

### SUMMARY OF THE INVENTION

Embodiments of the devices of the present invention provide a generally rugged and generally simple to set-up and use positioning apparatus. Such devices can be used to position and hold any appropriate instrument in the surgical field. Embodiments that are mechanical are generally rugged, require no utilities, and are easily set-up, cleaned, and sterilized.

The devices of the present invention include a control mechanism and a positioning mechanism. In some embodiments, the control mechanism and positioning mechanism are connected together by a mechanical means for transmitting force from the control handle to the positioning mechanism. In some embodiments the connection is a hydraulic system. In some embodiments, the hydraulic system is a closed-loop hydraulic system. In some embodiments the connection is a push-pull cable assembly. In some embodiments the connection is a system of cables and pulleys. In some embodiments the connection is made by two or more of a hydraulic system, a push-pull cable assembly, or a system of cables and pulleys. The control mechanism is located in a location generally convenient for the user. Movements of the control mechanism reposition the instrument because the positioning mechanism responds to the motion of the control mechanism, thereby repositioning the instrument to the desired location. In some embodiments the control mechanism is a handle. In some embodiments the control mechanism can be operated by the use of only one hand of the operator.

The devices of the present invention can have a variety of possible motion axes, or degrees of freedom, to achieve the desired control. In some embodiments, the device has two tilt axes and one extend axis. In some embodiments a first tilt axis allows the user to tilt the instrument forward or backward, thereby moving the tip of the instrument forward or backward. In some embodiments a second tilt axis tilts the tip of the instrument from side to side. The extend axis allows the user to extend or retract the tip of the instrument further in or out of the patient. In some embodiments, a rotate axis permits the user to rotate the instrument about its length. In some embodiments, the device includes additional motion axes, such as a grasp axis and a bend axis. The various axes described herein can be used in any combination in a particular embodiment.

In some embodiments, the positioning mechanism comprises a braking mechanism that can lock the positioning mechanism into a particular position, and wherein the control mechanism comprises an actuator for said braking mechanism.

In some embodiments, the positioning mechanism utilizes the tissue of the patient to create a pivot point for positioning of the instrument within the patient's body. In some embodiments, the positioning mechanism utilizes non-rigid pivot elements in positioning the instrument within the human body.

In some embodiments, the present invention includes methods of positioning an instrument for use in a surgical procedure. In some embodiments, these methods include methods of using the claimed devices to position an instrument for use during a surgical procedure. In some embodiments, the methods permit the surgeon to use only one hand to position an instrument for use during a surgical procedure.

### BRIEF DESCRIPTION OF THE DRAWINGS

The features, objects and advantages of the present invention will become more apparent from the detailed description set forth below when taken in conjunction with the drawings in which like references identify correspondingly throughout, and wherein:

FIG. 1 shows a perspective view of an embodiment of the present invention used in conjunction with various surgical devices during a surgical procedure.

FIG. 2 shows a schematic view of an embodiment of the positioning mechanism and an embodiment of the control mechanism connected by a mechanical force-transmitting connector.

FIG. 3 shows a schematic view of an embodiment of the positioning mechanism and an embodiment of the control mechanism connected by a hydraulic mechanical-force-transmission connector.

FIGS. 4a-4c show a schematic view of an embodiment of a closed-loop hydraulic system.

FIGS. 5a-f show a schematic view of the relationship between motions of an embodiment of the control mechanism and an embodiment of the positioning mechanism.

FIGS. 6a-c show a close-up schematic view of an embodiment of the positioning mechanism.

FIG. 7 shows a schematic view of an embodiment of the positioning mechanism and an embodiment of the control mechanism connected by a push-pull cable mechanical-force-transmission connector.

FIG. 8 shows a close-up schematic view of an embodiment of the control mechanism that utilizes a push-pull cable mechanical-force-transmission connector.

FIG. 9 shows a close-up schematic view of an embodiment of the positioning mechanism that utilizes a push-pull cable mechanical-force-transmission connector.

FIG. 10 shows a schematic view of an embodiment of the positioning mechanism and an embodiment of the control mechanism connected by a system of cables and pulleys.

FIGS. 11a-c show a close-up view of an embodiment of the control mechanism that has an embodiment of a brake system.

### DETAILED DESCRIPTION OF EMBODIMENTS

Certain embodiments of the invention will now be described with reference to the figures.

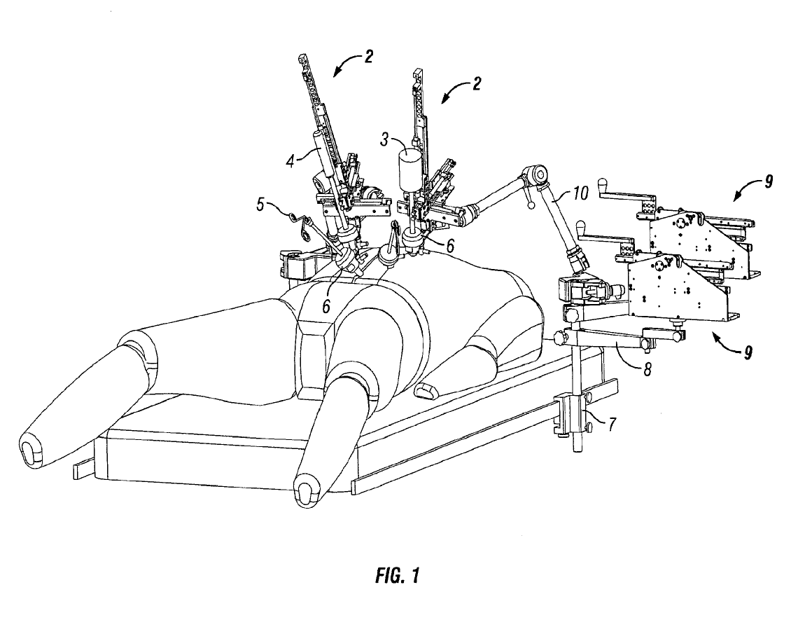

Referring to FIG. 1, numerous surgical devices are shown inserted into a patient on an operating bed. Laparoscopic instruments 5 are inserted through access ports 6 to cut, suture, manipulate tissue, etc. An endoscope/camera assembly 3, used to visualize the surgical site, is also inserted through an access port 6, and is held in place by the positioning mechanism 2. The positioning mechanism 2 is held by an adjustable arm 10, which is mounted on a support structure 7. A control handle 9 is mounted on a support bracket 8. In use, the user controls the position of the endoscope/camera 3 by manipulating the control handle 9, which causes the positioning mechanism 2 to move the endoscope/camera 3 to the desired position. Once the user stops manipulating the control handle 9 the positioning mechanism 2 stops moving and holds the endoscope/camera 3 in the new position.

Other instruments can also be positioned and held in this way. For example, a retractor 4 is shown attached to a positioning mechanism 2 in the same way as the endoscope/camera. The retractor 4 is pushed against organs or tissue to hold them out of the surgeon's way. The user manipulates the appropriate control handle 9 to cause the positioning mechanism 2 to move the retractor 4 in the appropriate direction. Once the user stops moving the control handle 9 the positioning mechanism 2 stops moving and holds the retractor 4 in the desired position. Of course any other instrument useful in a surgical procedure could be held and manipulated by embodiments of the devices of the present invention. The variety of devices which can be thus moved and held by the positioning mechanism and control handle are referred to below as “instrument(s)”. The instruments may be permanently coupled to the positioning mechanism 2 or interchangeable attached. In some embodiments, an instrument is coupled to the positioning mechanism 2 prior to the instrument's insertion into the patient's body. In other embodiments, the instrument is first manually inserted into the body and positioned followed by coupling to the positioning mechanism 2. In some embodiments, the positioning mechanism is located outside of the patient's body and couples to an instrument outside of the patient's body.

With the positioning mechanism 2 and control handle 9 arrangement described above the surgeon can reposition and hold various instruments without the need for an assistant—thereby avoiding the problems of communicating with that assistant, or the problems of fatigue and loss of attention of the assistant.

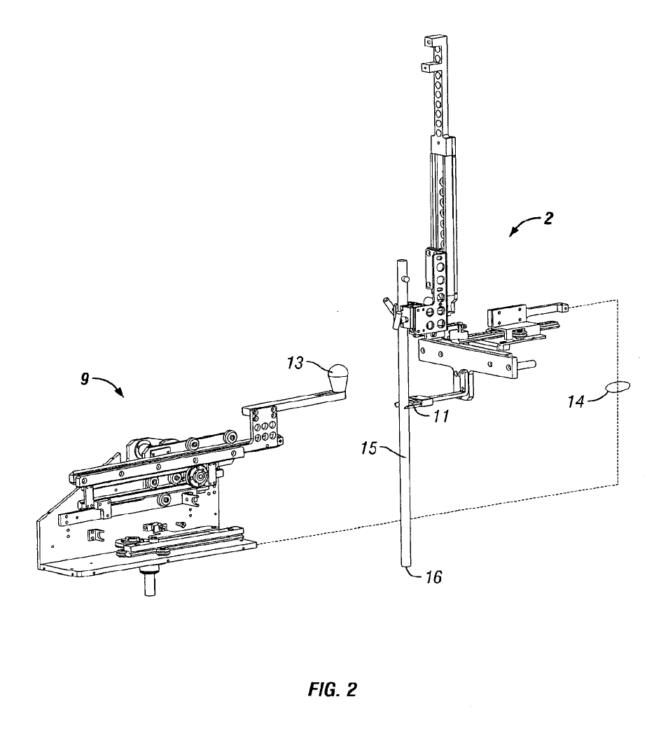

FIG. 2 shows an embodiment of the positioning mechanism 2 and an embodiment of the control mechanism, control handle 9, connected by a mechanical force-transmitting connector 14. This mechanical force-transmitting connector 14 transmits force signals from the control handle 9 to the position mechanism 2, allowing the user to move the positioning mechanism 2 by manipulating the control handle 9. As discussed below, the mechanical force-transmitting connector 14 can be hydraulic, cable-pulley, push-pull cable, or other mechanical means.

The control mechanism can have any configuration which permits the surgeon to effectively manipulate the positioning mechanism. In the depicted embodiment, the control mechanism is a particular control handle 9. However, other control mechanisms are contemplated. By way of non-limiting example, the control mechanism may have a glove-like configuration that engages the users arm, hand, and fingers.

In use, the user moves the control handle 9 by pushing knob 13 in the desired direction. Force signals are transmitted from the control handle 9 to the positioning mechanism 2 via the mechanical force-transmitting connector 14, causing the positioning mechanism 2 to move in response. The instrument 15 moves in several axes. In one embodiment the instrument pivots about the point 11 where it enters the patient. The patient's tissue at point 11 can serve as the pivot, or a pivot bearing (not shown) can be provided to cause the instrument 15 to pivot about point 11. The positioning mechanism 2 pushes the instrument 15 forward-backward, side-to-side, or any combination of these two. The instrument 15, constrained at point 11 by either the patient's tissue or a pivot bearing (not shown), tilts about point 11, with the result that the distal tip of the instrument 16 moves to a new position inside of the patient. One embodiment also contains an extend axis which permits the user to extend or retract the distal end of the instrument 16.

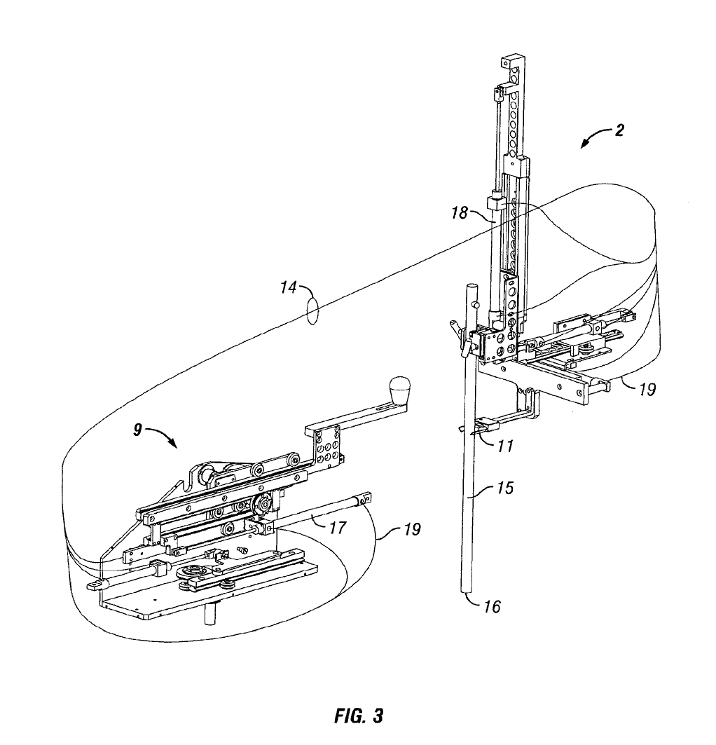

Referring to FIG. 3, one embodiment is shown in which the mechanical-force-transmission connection is hydraulic. Motions of the control handle 9 cause hydraulic fluid (not shown) to travel through tubing to the positioning mechanism 2, which responds to tilt and/or extend/retract the instrument 15 about point 11, thereby repositioning the distal tip 16 of the instrument 15 inside the patient. Conventional hydraulic systems, employing cylinders, pumps, valves, and reservoirs can be used. A hydraulic method is shown in FIG. 3. Control hydraulic cylinder(s) 17 in the control handle 9 are connected in a closed-loop circuit to slave hydraulic cylinder(s) 18 in the positioning mechanism 2 via tubing 19. When the user moves the control handle 9 to a new position, the shaft of the control cylinder 17 is pushed or pulled, thereby displacing hydraulic fluid in the control cylinder 17. This hydraulic fluid is forced through tubing 19 to the responding slave cylinder 18 in the positioning mechanism 2, causing the shaft of the slave cylinder 18 to move. This movement is used to tilt and/or extend/retract the instrument.

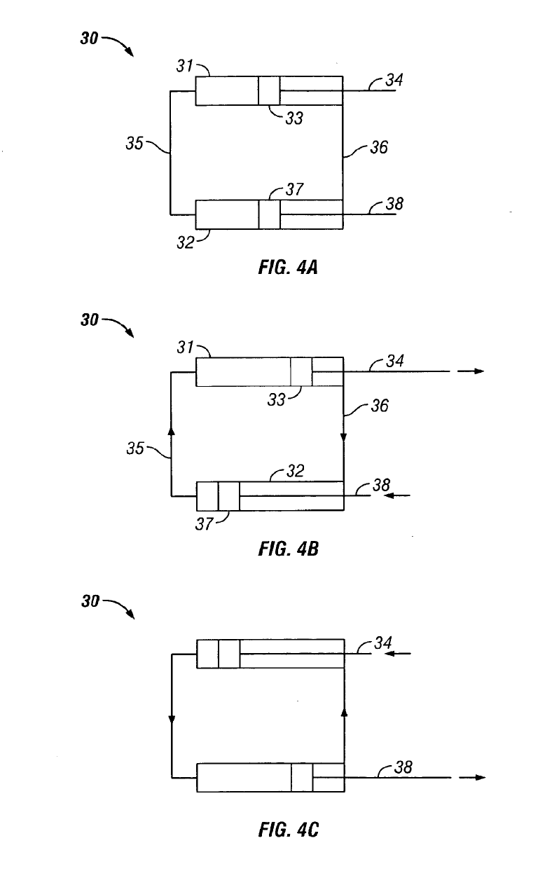

FIGS. 4a-4c. show this action in schematic form. A basic closed-loop hydraulic circuit 30 is shown in FIG. 4a. The control cylinder 31 contains a piston 33 which is connected to a shaft 34. Similarly, the slave cylinder 32 contains a piston 37 connected to a shaft 38. The back side of each cylinder is connected to the other by tubing 35. Similarly, the front side of each cylinder is connected to the front of the other by means of tubing 36.

As shown in FIG. 4b, the shaft 34 of the control cylinder 31, located in the control handle 9, is pulled to the right, pulling the piston 33 to the right. This action causes hydraulic fluid to travel from the front of control cylinder 31 to the front of slave cylinder 32 via tubing 36. This forces the shaft 38 and piston 37 in slave cylinder 32 to move to the left. This drives hydraulic fluid from the back of slave cylinder 32 to the back of control cylinder 31 via tubing 35. The motion of slave shaft 38 is used in the positioning mechanism 2 to reposition the tip 16 of the instrument to the desired location.

FIG. 4c shows the reverse motion, in which the control shaft 34 is moved to the left, causing the slave shaft 38 to move to the right.

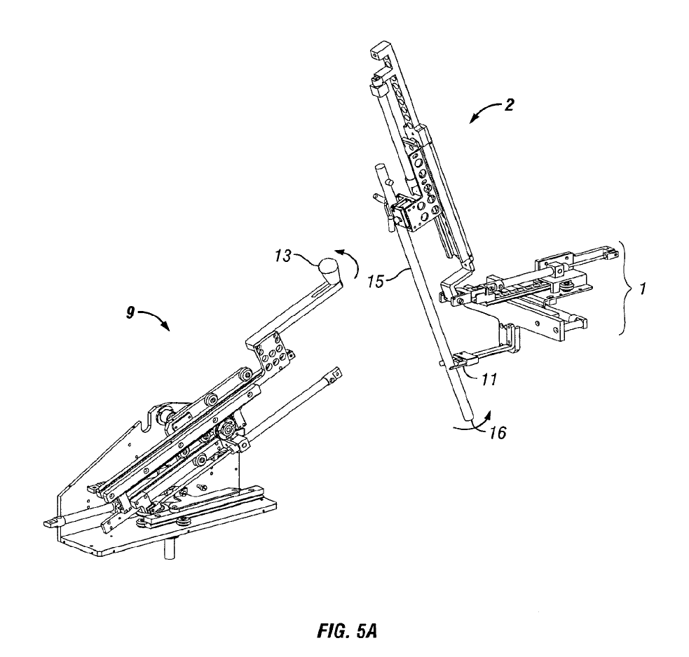

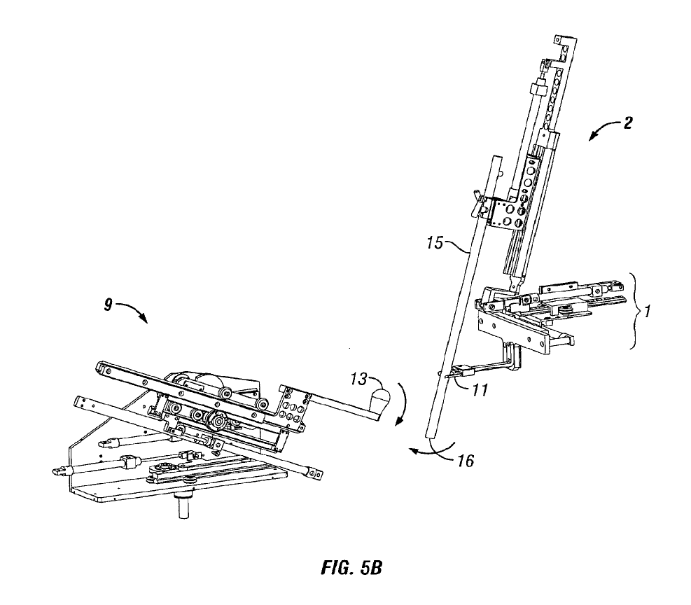

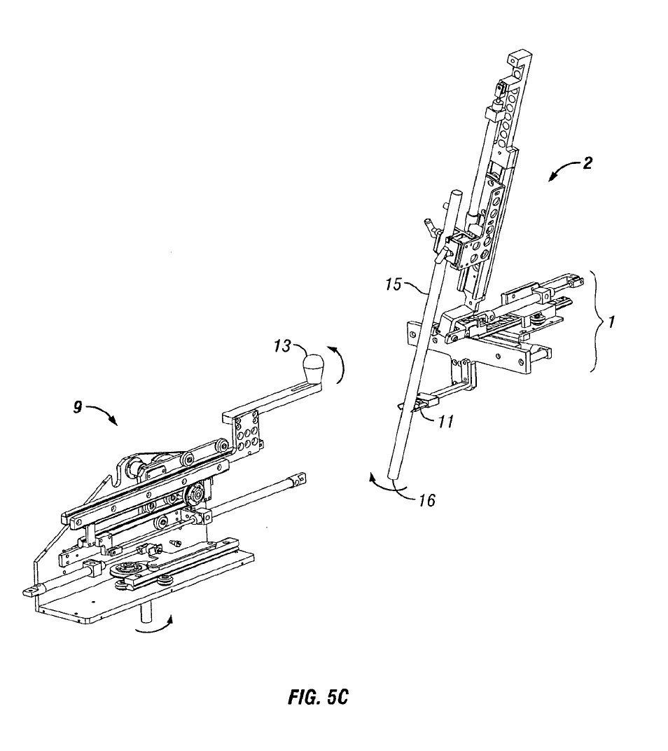

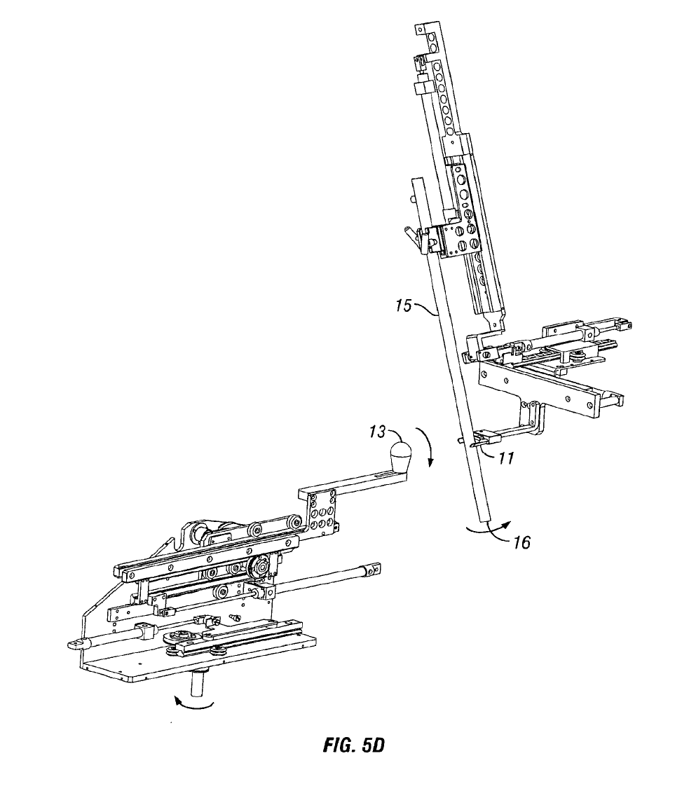

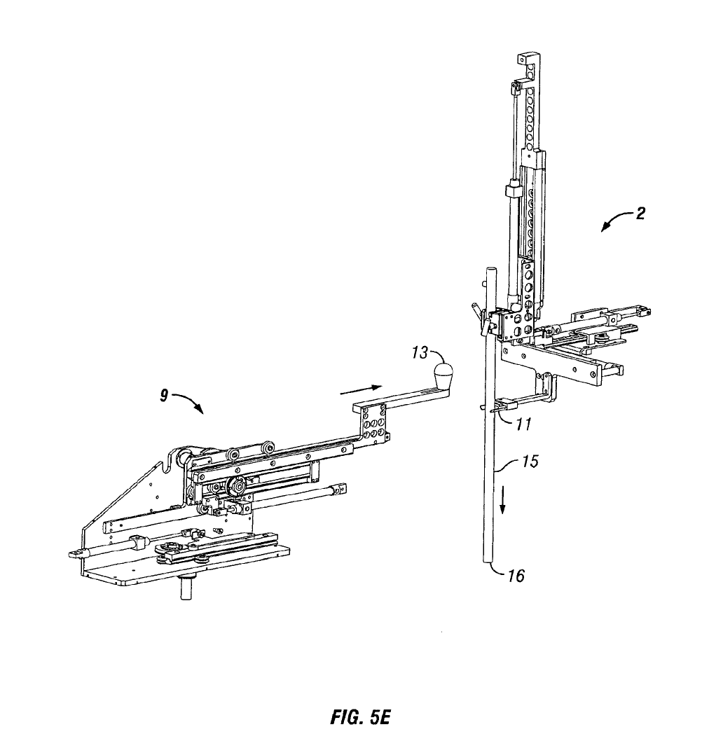

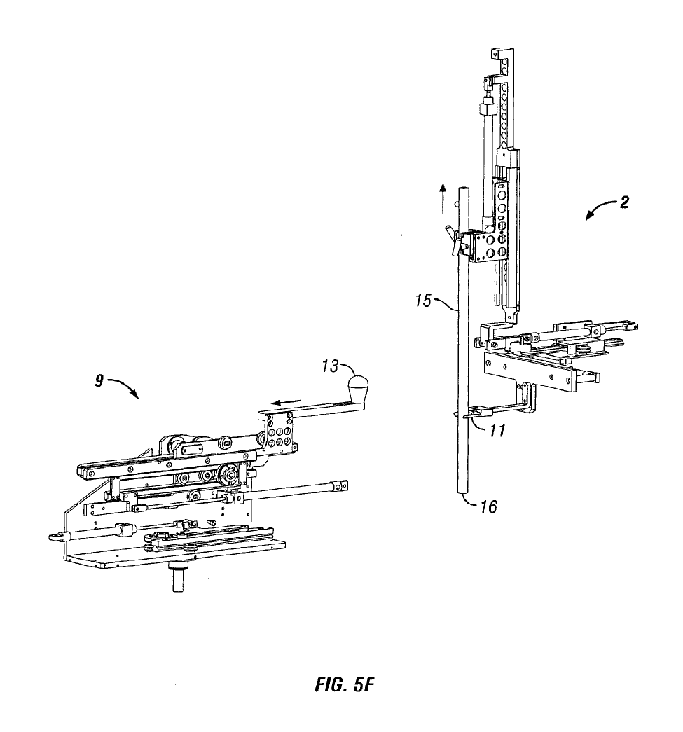

FIGS. 5a-f show the relationship between motions of the control handle 9 and an embodiment of the positioning mechanism 2. In FIG. 5a the knob 13 of control handle 9 has been pulled upward, forcing hydraulic fluid to travel between control cylinders in control handle 9 and slave cylinders in positioning mechanism 2, thereby causing positioning mechanism 2 to tilt the instrument 15 about point 11 and thus move the distal tip 16 of instrument 15 back in relation to the housing 1 of the positioning mechanism 2. FIG. 5b similarly shows the knob 13 pushed downward, causing tip 16 to move away from the housing 1 of positioning mechanism 2. FIG. 5c shows the knob 13 moved to the left, thereby driving tip 16 to the right relative to housing 1 of positioning mechanism 2. Similarly FIG. 5d shows the knob 13 moved to the right, thereby driving tip 16 to the left relative to housing 1 of positioning mechanism 2. In FIG. 5e the knob 13 is pushed forward to extend tip 16 further into the patient, and similarly FIG. 5f shows the knob pulled backward to retract tip 16 from the patient.

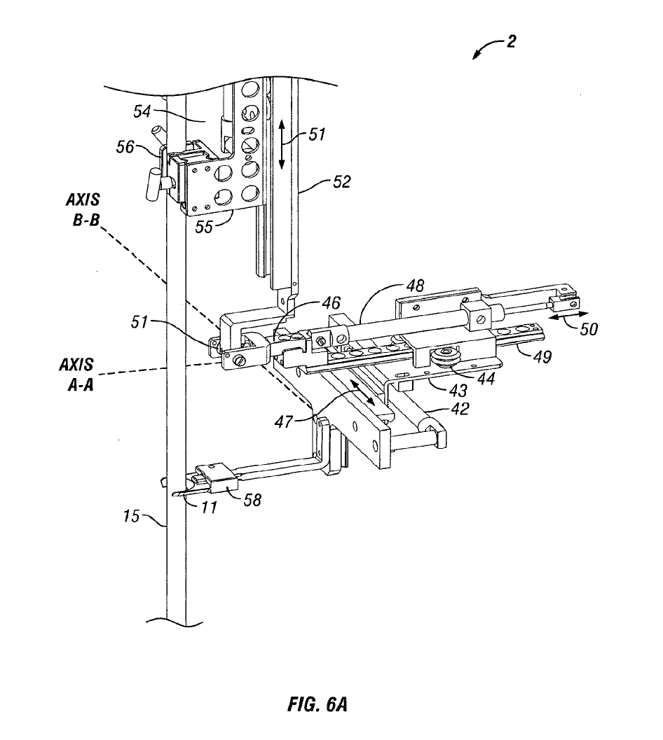

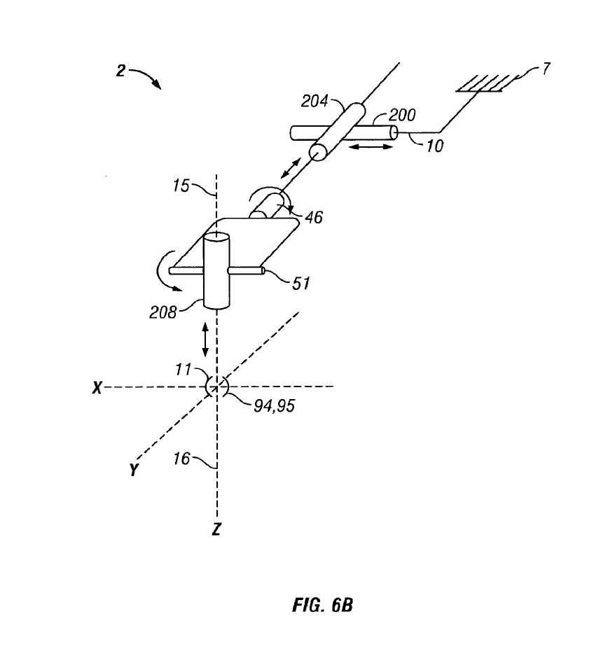

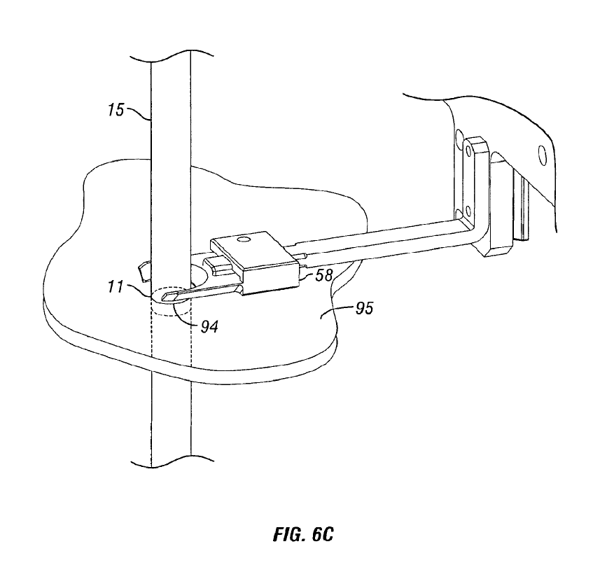

Referring to FIG. 6a, more detail of an embodiment of the positioning mechanism is provided. All three of the motion axes comprise a slave cylinder and guide device. The side-to-side motion is achieved by motion of slave cylinder 42, which pushes/pulls tilt slide assembly 44, which is free to move side-to-side as shown by arrow 47. This motion is transmitted to instrument slide assembly 52 by a non-rigid pivot bearing 46. This pivot bearing 46 allows the instrument slide assembly 52 to rotate about axis A-A and automatically assume the correct angle to permit the instrument 15 to pivot about point 11. The forward/backward motion is achieved by motion of slave cylinder 48, which pushes and pulls guide device 49 along rollers 44 as shown by arrow 50. The motion of guide device 49 is transmitted to instrument slide assembly 52 via non-rigid pivot bearing 51. This pivot bearing 51 allows the instrument slide assembly 52 to rotate about axis B-B and automatically assume the correct angle to permit the instrument 15 to pivot about point 11. The extend/retract motion is achieved by motion of slave cylinder 54, which pushes/pulls extend slide 55 in the direction indicated by arrow 57. Instrument 15 is attached to extend slide 55 by clamp 56, and thus extended or retracted in the patient.

FIG. 6b shows a schematic depiction that more clearly shows the movable elements of an embodiment of the positioning mechanism 2. In the depicted embodiment, the mechanism consists of a novel arrangement of three sliders, two rotating joints, and one spherical joint. A first slider 200 is mounted on adjustable arm 10, connected to support structure 7. A second slider 204 is mounted on first slider 200. A first rotating joint 46 is mounted on the second slider 204. A second rotating joint 51 is mounted on first rotating joint 46. A third slider 208 is mounted on second rotating joint 51. Spherical joint 210 is formed by the incision 94 in the patient's tissue 95 (as depicted in FIG. 6C). The transverse motion of first slider 200 is transmitted, via second slider 204 and first (46) and second (51) rotating joints, to third slider 208. This motion causes instrument 15 to pivot about incision 94, driving distal tip 16 in a direction opposite to the movement of the first slider. Similarly, transverse motion on second slider 204 is transmitted via first (46) and second (51) rotating joints to third slider 208. This motion causes instrument 15 to pivot about incision 94, driving distal tip 16 in a direction opposite to the movement of the second slider 204. Transverse motion of third slider 208 either extends the instrument 15 further into incision 94 or retracts the instrument further out of incision 94.

Because non-rigid pivot bearings 46 and 51 are free to move, a second pivot device is required at point 11 to force the instrument to pivot about this point. In one embodiment the tissue of the patient acts as a pivot bearing, allowing instrument 15 to tilt about point 11. This embodiment is shown most clearly in FIG. 6C. In order to aid the user in locating the positioning mechanism 2 optimally over the incision 94 at point 11 in the patient tissue 95, a guide shoe 58 is provided. During setup the user locates the center of the shoe 58 over the incision 94 at point 11, then inserts the instrument 15 into the incision 94 in patient tissue 95, and attaches it to the extend slide 55 with clamp 56. Such a setup is depicted in FIG. 6A. In another embodiment a spherical bearing (not shown) is provided to create the second pivot bearing, which would be located over the incision at point 11 as well.

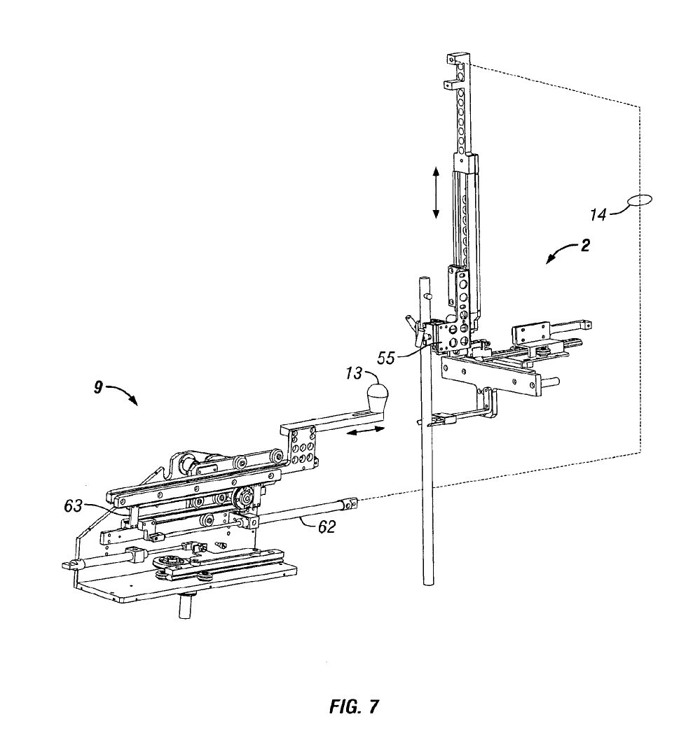

Referring to FIG. 7, an alternative embodiment is shown. In this embodiment, the mechanical force transmission connector 14 is a system of push-pull cable assemblies. Basic push-pull cable assemblies are well known in the art. Generally, push-pull cable assemblies comprise a flexible cable carried within a flexible guide tube. By pushing or pulling on one end of the cable, motion is transmitted to the other end of the cable, as is commonly seen in bicycle gear changing mechanisms. By example, in FIG. 7 the extend axis is shown driven by a push-pull cable assembly 62 which is attached to the extend mechanism 63 in control handle 9 and to the extend slide 55 in positioning mechanism 2. By pushing/pulling the knob 13 the cable in cable assembly 62 is pushed/pulled, causing the extend slide 55 in positioning mechanism 2 to move in response.

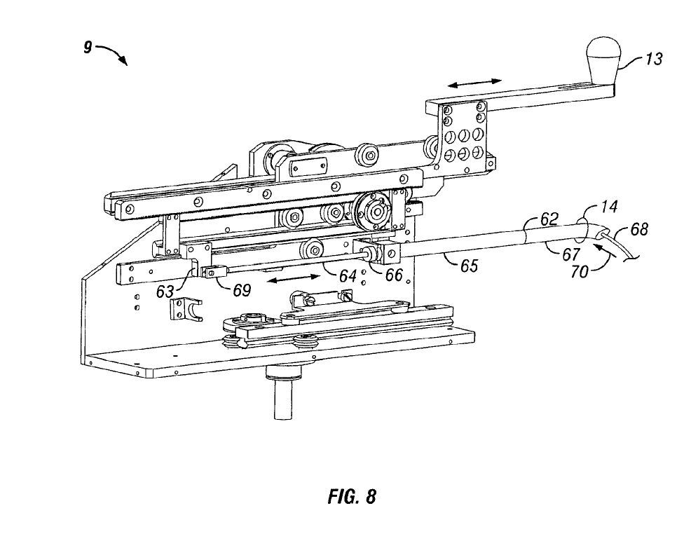

FIG. 8 shows more detail of the push-pull cable used in the extend axis of control handle 9. Push-pull assembly 62 comprises a rigid shaft 64 that is anchored to the extend mechanism 63 by coupling 69. As knob 13 is pushed-pulled, the extend mechanism 63 pushes or pulls on shaft 64 via coupling 69. Shaft 64 is pushed-pulled into housing 65. Within housing 65 the shaft 64 is connected to flexible cable 68, which slides within flexible guide 67. The resulting motion of cable 68 is indicated by arrow 70.

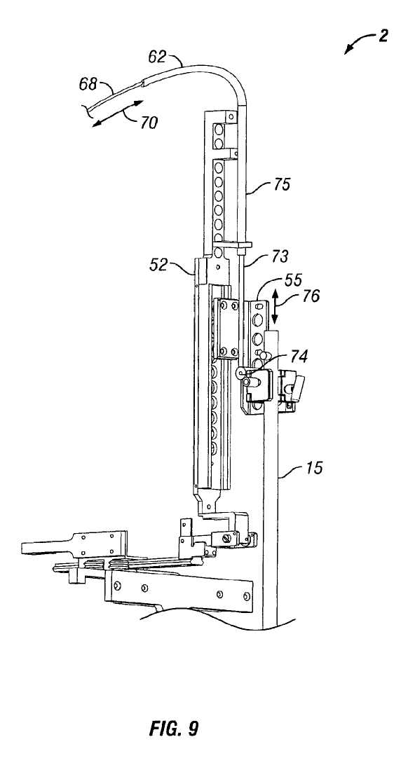

Referring now to FIG. 9, the cable assembly 62 terminates at the instrument slide assembly 52 of the positioning mechanism 2. The motion of the flexible cable 68, indicated by arrow 70, is transmitted to the extend slide 55 by rigid shaft 73. The resulting motion of extend slide 55 is indicated by arrow 76.

For clarity and simplicity FIGS. 7, 8, and 9 show only the extend axis driven by a push-pull cable assembly, but this invention contemplates that all motion axes described herein could be similarly be driven with push-pull cables.

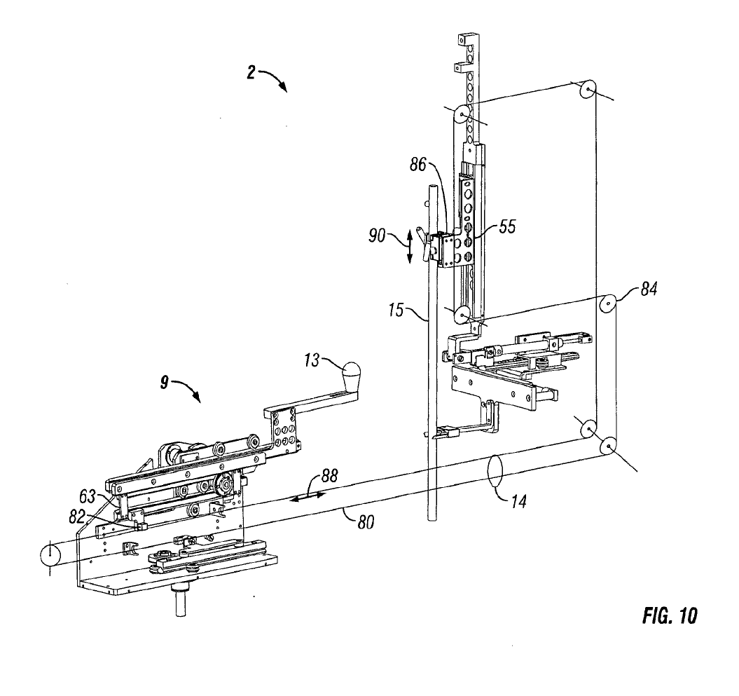

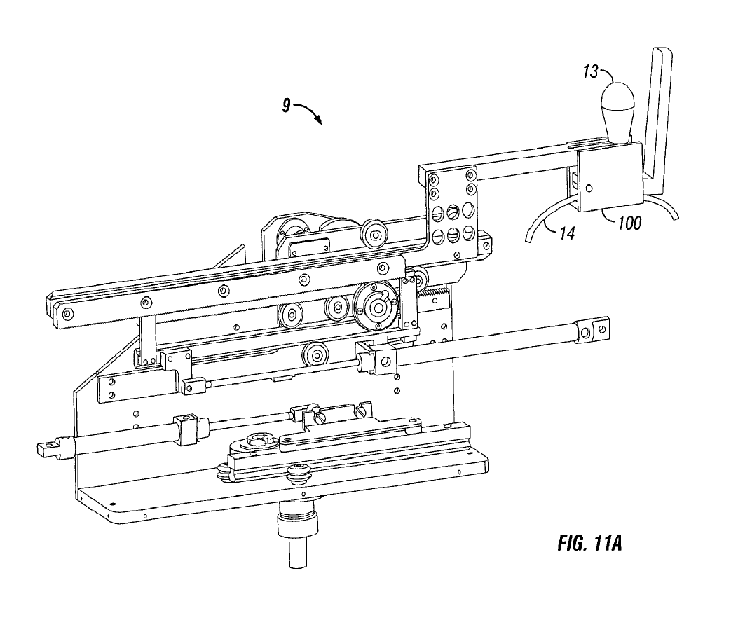

Another embodiment is shown in FIG. 10. In this embodiment the mechanical force transmission connector 14 is a system of cables and pulleys, shown in semi-schematic form. FIG. 10 depicts the extend axis driven by a cable/pulley arrangement. A flexible cable 80 is attached to the extend mechanism 63 on control handle 9 at coupling 82. Cable 80 is directed around several pulleys 84 to connect the extend mechanism 63 of the control handle 9 to the extend slide 55 on the positioning mechanism 2 at coupling 86. Motion of the extend mechanism 63 results in motion of the cable 80 as shown by arrow 88. This motion is transmitted to the extend slide 55 by cable 80, resulting in motion of the instrument 15 shown by arrow 90.

For clarity and simplicity FIG. 10 shows only the extend axis driven by a cable/pulley arrangement, but this invention contemplates that all motion axes described herein could be similarly driven with cable/pulley arrangements.

This invention also contemplates the use of other mechanical force transmission connections. For example, this invention includes devices utilizing rigid rods connected by universal joints and couplings, push-pull tapes, belts, chains, and ball drives.

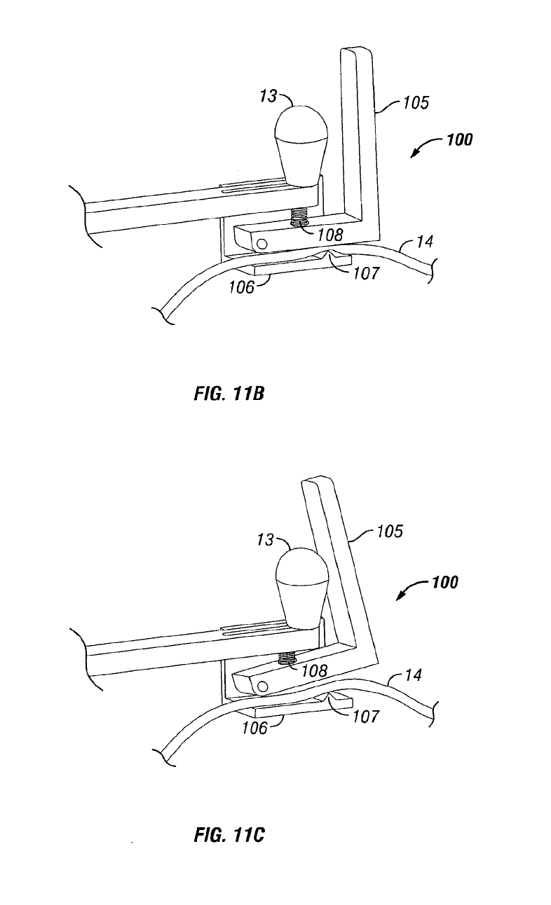

Other embodiments are illustrated in FIGS. 11a-b. Referring to FIG. 11a, a brake mechanism 100 is shown attached to the control handle 9. In the depicted embodiment, the brake 100 is normally on, i.e. the brake is active and preventing motion, unless deactivated by the user. To reposition the instrument, the user grasps the brake mechanism 100, applies force to deactivate the brake, and repositions the instrument. When the new position is reached the user releases the brake mechanism 100, thus reactivating the brake.

Referring to FIG. 11b, an embodiment of the brake mechanism 100 is shown, with one wall removed for clarity, in the actuated position. In this embodiment, the mechanical force transmission connector is hydraulic, but it is contemplated that a brake mechanism could be used with embodiments having any mechanical force transmission connector (for example, one utilizing push-pull cables or cable and pulley systems). In this embodiment, hydraulic tubing 14 (only one tube is shown for clarity) is pinched between pinch point 107 on brake housing 106 and brake lever 105 due to force applied by spring 108. Flow of hydraulic fluid through tubing 14 is thereby prevented, thus preventing motion of the instrument.

FIG. 11b shows an embodiment of the brake mechanism 100 in the deactivated position. Again, in this embodiment, the mechanical force transmission connector is hydraulic, but it is contemplated that a brake mechanism could be used with embodiments having any mechanical force transmission connector (for example, one utilizing push-pull cables or cable and pulley systems). The brake lever 105 has been pulled back toward knob 13, compressing spring 108 and causing brake lever 105 to rotate away from pinch point 107, thereby releasing pressure on, and allowing flow through, tubing 14. In this position motion is allowed and the instrument can be repositioned.

Embodiments of the invention include surgical devices and components coupled with surgical devices. It is appreciated that the surgical devices and other components described in conjunction with the present invention may be electrically, mechanically, hydraulically, directly, indirectly and remotely coupled. It is appreciated that there may be one or more intermediary components for coupling components that may or may not be described.

For example, telemanipulation and like terms such as “robotic” refer to manipulating a master device and translating movement or force applied at the master device into commands that are processed and transmitted to a slave device that receives the commands and attempts to generate the intended movements at the slave device. It is appreciated that when using a telemanipulation device or environment, the master and slave devices can be in different locations.

Embodiments of the present invention are well suited to be used with both telemanipulation systems direct manipulation systems. It is also appreciated that embodiments of the present invention are well suited to be used inside and outside a body.

In one embodiment, embodiments of the present invention described above may further comprise an end effector coupled to the output end of the plurality of couplings, wherein the end effector moves in response to receiving at least the portion of the input force transmitted by the plurality of couplings. Optionally, the end effector comprises a surgical tool. It is appreciated that the input force may be generated by a direct manipulation device or may be generated by a telemanipulation device.

In yet another aspect, the present invention may further comprise a manually-driven hydraulic drive system having an input mechanism coupled to the input end of the plurality of couplings, wherein the drive system generates the input force, and an end effector coupled to the output end of the plurality of couplings, wherein the end effector comprises a surgical tool and moves in response to receiving at least the portion of the input force transmitted by the plurality of couplings. It is appreciated that the input force may be generated by a direct manipulation device or may be generated by a telemanipulation device.

The present invention relates to surgical tools and surgical devices that can be used inside and outside a body. For illustrative purposes, these aspects are discussed herein with respect to a surgical application, however, it should be understood that these aspect may equally apply to many other applications, such as robotics, manufacturing, remote controlled operations, etc., and any application where the tool holding and tool positioning devices of the present invention can be used.

Aspects of the present invention include features relating to tool holding and tool positioning devices for surgical-related activities and methods of manufacture and use thereof, including variations having an angularly moveable hub housing and a rotatable and operable end effector driven via additional drive train elements that include one or more flexible couplings, such as universal-type joints. Force transmitted via the set of such elements includes, for example, lineal force and rotational force. It is appreciated that the force transmitted may be generated locally or remotely to the output device and it should be appreciated that embodiments of the present invention are well suited to be used in both direct manipulation and telemanipulation environments.

In one variation, aspects of the present invention include a push-pull-rotate (PPR) element that permits the transmission of axial forces and angular torques around corners or bends. The PPR element may include one or more universal joints (e.g., Hooke's joints) or similarly operating mechanisms arranged in series (in a chain-like configuration) and connected to an input and to an output. The PPR element may be contained within a housing. It is appreciated that the input and/or output may be coupled with a remote telemanipulation device or may be coupled to a direct manipulation device and can be used in both direct manipulation environments and telemanipulation environments.

In some embodiments, a guide element is provided to prevent portions of the PPR element from collapsing under compression and to maintain proper form under extension, among other things. Exemplary motion that may be transmitted to the end effector and/or tools via the PPR element may include rotational motion and push-pull or reciprocating motion that may be used, for example, to cause two or more extensions of the end effector to move relative to one another (e.g., to open and close to allow grasping or cutting, and release). It is appreciated that the exemplary motion may be initiated by a direct manipulation or a telemanipulation input force. It is appreciated that the input force to induce the exemplary motion may be generated in a remote location wherein the input device and output device are coupled with a telemanipulation system.

In one variation, the guide element is responsive to the bend angle and is adjusted appropriately or automatically adjusts its position as a function of operation of the device within a motion limiting mechanism, such as a guide track into which an extension from the guide element slides. The bending of the device to various bend angles may be accomplished via use of one or more pivot points and control mechanisms, such as tendon-like linkages. The PPR element may be attached to a source or sources of axial and torsional input (also interchangeably referred to herein as an “input mechanism”), such as a rotatable and extendable and retractable shaft, housed in a body portion. It is appreciated that the source input may be from a direct manipulation or a telemanipulation input force.

Axial and torsional inputs to each of the PPR elements are then transmitted from the PPR elements to any output, such as to permit rotation and operation of an end effector. The end effector may rotate, for example, along with a PPR element via a sleeve. It is appreciated that the input may be separated from the output by a telemanipulation system where the force is transmitted from the input to the output via a telemanipulation system.

Some variations of the present invention use one or more essentially friction-free or low friction components in the PPR element and guide system, such as rolling-element bearings, which results in relatively high mechanical efficiencies (e.g., as compared to push-pull cables or cable-pulley systems). Other portions of the system relating to movement, such as guide track pins and pivots in some variations, can optionally be replaced with or further include low-friction rolling-element bearings for even smoother action. Appropriate guide track, guide housing, and hub or rotating tip components can comprise non-conductive material to manage the distribution of electrical energy to end-effectors. Any components may be plated with an appropriate anti-friction and/or electrically insulating coating and/or be used with suitable lubricating substance or features.

Conversely or in addition, some portions of the system may be electrically conductive, such as for use in electrosurgery applications. For example the outer housing of the device may be non-conductive, so as to insulate inner conductive portions. The motion transmitting inner portions may be conductive so as to allow electrosurgical current to be delivered to the end effector and/or any tools used therewith, while the outer housing thereby insulates the device. In addition to certain components being conductive, conducting lubricants may also be used to ensure or enhance electrical communication. In some variations, the electrical energy communicated may be of high frequency to enhance communication of the energy across abutting surfaces and lubricants. It is appreciated that in one embodiment, the electrical communication may be generated from a telemanipulation system.

Aspects of the present invention relate to interchangeable tools for use within a closed area. In general, disclosed herein is a holder which comprises one or more tools attached thereto. The holder and the attached tools are so configured that they can be inserted into a closed area and easily manipulated therein. Examples of the closed area include inside the body of a patient, as in during laparoscopic or arthroscopic surgery, or inside of a device or a mechanical object, as in during maintenance or repair of the interior of said device or mechanical object.

In one embodiment, the tools are configured to be attached to the distal end of a manipulator, which itself is configured to receive the tools. The distal end of the manipulator can itself be inserted into the closed area. The distal end of the manipulator can be controlled by an operator at a proximal end, i.e., the end closest to the operator. It is appreciated that in one embodiment, the proximal end and operator may be remote to the distal end may be coupled with a telemanipulation system that allows the operator to provide input forces remotely to the patient.

Within the closed area, the operator can choose a desired tool from a selection of tools on the holder and attach it to the distal end of the manipulator. After the operator has used the tool in a desired fashion, the operator can then return the just-used tool to the holder, obtain a second tool from the holder, attach it to the distal end of the manipulator, and use the second tool. The operator can repeat this process as many times as the operator desires, thereby interchanging the tool used inside the closed area without having the need to withdraw the manipulator from the closed area. In one embodiment, the operator can change tools within the patient from a remote location.

As described in detail, this system is designed for use, for example, in laparoscopic surgery. The tools are various surgical tools used within the patient's body. The tools in the holder are inserted into the body. During surgery, the surgeon can use and exchange tools without the need to remove the manipulator or the tools themselves from the body. This represents a significant improvement over existing methods and devices. It is appreciated that in one embodiment, the operator can change tools within the patient even in the case that the operator is remote to the patient. In this embodiment, a telemanipulation system may be used to couple the input end with the output end.

A “manipulator” as used herein refers to a device that at its proximal end comprises a set of controls to be used by an operator and at its distal end comprises means for holding and operating a tool, referred to herein as the “tool receiving device.” The controls allow the operator to move the tool receiving device within the generally closed or confined area, and operate the tool as intended. The tool receiving device is adapted to receive tools interchangeably and can cause a variety of different tools to operate in their intended purpose. Examples of a manipulator include any of a variety of laparoscopic or arthroscopic surgical tools available on the market for use by surgeons, or the device described in U.S. Pat. No. 6,607,475. The tool receiving device of a manipulator is adapted to enter a generally closed or confined area through a small opening, such as a small hole in a mechanical device or a small incision in a human body. It is appreciated that the proximal end may be remote to the distal end and can be used in a telemanipulation environment.

As used herein, “proximal” refers to the part of the device that remains outside of the closed area, closest to the operator. “Distal” refers to the end inserted into the closed area, farthest away from the operator. The proximal and distal ends are preferably in communication with each other, such as fluid communication, electrical communication, communication by cables, telemanipulation and the like. Such communication can occur, for example, through a catheter or cannula, which houses the lines used for such communication. The catheter or cannula is preferably a tube or other substantially cylindrical hollow object. In some embodiments, the catheter or cannula does not house any lines for communication between the proximal and distal ends. In these embodiments, the catheter or cannula is used for placing an object, located substantially at the distal end of the catheter or cannula, inside the closed area for further manipulation. It is appreciated that the distal and proximal ends may be in communication with the use of a telemanipulation system.

During the operation of the devices described herein, the catheter or cannula (hereinafter referred to simply as “cannula”) is inserted into a generally closed or confined area where the tools are to be used such that its proximal end remains outside the closed area while the distal end remains inside the closed area. In the context of surgical procedures, the cannula is inserted into the patient's body such that its proximal end remains outside the body while the distal end remains inside the body. In one embodiment, the proximal end is remote to the patient. This allows the operator, e.g. a surgeon, to access the interior of the closed area, e.g., a patient's body, using the cannula, thereby eliminating the need for “open” surgical procedures both locally and remotely. Only a small incision is needed to insert the cannula, and the various surgical instruments are inserted, and the procedures performed, through the cannula. The proximal end may be remote to the patient and force applied at the proximal end may be translated using a telemanipulation system that recreates the input force at the distal end.

The instruments or tools described herein are capable of being attached to the distal end of the manipulator in a number of different ways. For instance, in some embodiments the tools are attached magnetically, while in other embodiments the tools may clip on to the distal end of the manipulator. In one embodiment, a telemanipulation system may be used to couple the distal and proximal ends. Additional details on the attachment of the tools is provided below.

The manipulator, which is used to position and maneuver the tools within the confined space, can be a hydraulic, pneumatic, robotic, direct manipulation, telemanipulation, standard surgical, minimal invasive surgery (MIS), electrical, or mechanical device, or a device comprising a combination of any of these systems. Any system that can be used to position and manipulate the tools is contemplated.

What is claimed is:

1. A device for use in positioning an instrument for use in a surgical procedure, comprising:
a positioning mechanism configured to couple to the instrument and to move the instrument relative to the patient's body;
a control mechanism; and
a connector operatively coupled to the control mechanism and the positioning mechanism, wherein the control mechanism is configured to cause the positioning mechanism to move the instrument by transmitting force to the control mechanism through the connector.

2. The device of claim 1 wherein said force is a human applied force.

3. The device of claim 1 wherein said positioning mechanism couples to said instrument outside said patients body.

4. The device of claim 1 wherein said positioning mechanism couples to said instrument inside said patients body.

5. The device of claim 1, wherein said control mechanism is remote to said positioning mechanism.

6. The device of claim 1 wherein said connector includes a telemanipulation device.

7. The device of claim 1, wherein the connector comprises a hydraulic system.

8. The device of claim 7, wherein the hydraulic system comprises a closed-loop hydraulic system.

9. The device of claim 1, wherein the connector comprises a push-pull cable system.

10. The device of claim 1, wherein the connector comprises a cable and pulley system.

11. The device of claim 1, wherein the connector includes more than one of a hydraulic system, a push-pull cable system, a telemanipulation system and a cable and pulley system.

12. A device for use in positioning an instrument for use in a surgical procedure, comprising:
a positioning mechanism coupled to a support structure, wherein the positioning mechanism and support structure are located outside of a patient's body;
a surgical instrument coupled to the positioning mechanism and extending into the patient's body;
a control mechanism; and
a connector coupled to the control mechanism and the positioning mechanism, wherein the control mechanism is configured to cause the positioning mechanism to move the instrument relative to the patient's body by transmitting control signals through the connector.

13. The device of claim 12 wherein said connector includes a telemanipulation device.

14. The device of claim 12 wherein said control mechanism is remote to said positioning mechanism.

15. The device of claim 12 wherein said control signals are generated by a telemanipulation device.

16. A method of positioning relative to a patient an instrument for use in a surgical procedure, the method comprising:
securing a positioning mechanism to a support structure;
inserting the instrument into the patient's body, wherein the instrument is coupled to the positioning mechanism; and
manipulating a control mechanism coupled to the positioning mechanism, wherein manipulation of the control mechanism causes the positioning mechanism to move the instrument relative to the patient's body.

17. The method of claim 16 further comprising:
manipulating said control mechanism remotely to said positioning mechanism using a telemanipulation device.

18. The method of claim 16 further comprising:
coupling said positioning system with said control system with a telemanipulation device.

19. The method of claim 16 wherein the positioning mechanism and support structure are secured outside said patient's body.
